(19) United States
(12) Patent Application Publication
(10) Pub. No.: US 2005/0100903 A1
(43) Pub. Date: May 12, 2005

(54) Screening method

(76) Inventor: Michael Neumaier, Edingen-Neckarhausen (DE)

Correspondence Address: WHITHAM, CURTIS & CHRISTOFFERSON, P.C., 11491 SUNSET HILLS ROAD, SUITE 340, RESTON, VA, 20190, US

(21) Appl. No.: 10/482,107

(22) PCT Filed: Jun. 28, 2002
(86) PCT No.: PCT/EP02/07185

(30) Foreign Application Priority Data
Jun. 28, 2001 (DE) 101 31 382.9

(51) Int. Cl. C12Q 1/68; C12N 15/85; A61K 48/00

(52) U.S. Cl. 435/6; 435/455; 514/44

(57) ABSTRACT

The invention describes a method wherein therapeutically useful compounds can be identified via the determination of the expression of glycoprotein antigens of the CEACAM family. These have properties that can prevent the development of hyperplastic alterations that have been identified as the precursors of neoplastic transformation and can lead to the development of a carcinoma, or restore the normalization of the tissue. In particular, the present invention relates to a method for the identification of one or more compounds that are suitable for the prevention of tumorigenesis or for the treatment of precursor stages of tumors. The selected compounds are in a position, through regulation of gene expression, to increase the apoptosis sensitivity (or lowers the apoptosis resistance) of cells of the colon mucosa, especially the precursor cells. In addition, the invention is directed towards a diagnostic method and towards the use of cells identified in accordance with the invention in pharmaceutical compounds for the prevention of tumorigenesis and the treatment of precursor stages.

### SUBJECT OF THE INVENTION

The invention describes a method for the identification of one or more therapeutically useful compounds. This compound/compounds has/have characteristics that can prevent the genesis of the earliest morphological tissue alterations that can lead to the development of cancer, or to induce programmed cell death (apoptosis). As a result, the tissue situation is normalized.

### PRIOR ART

The development of tumors, adopting colon carcinoma as an example

Carcinomas develop in successive steps from normal tissue via intermediate forms (normal-to-tumor transition or multi-step carcinogenesis). Genetic alterations (mutations) have been described being related to this alteration. Multi-step carcinogenesis was formulated by the group headed by Bert Vogelstein (e.g. Fearon E. R. and Vogelstein B., Cell, 1990, 61, pages 759ff) and has been confirmed by many other groups in the literature (FIG. 1). This model states that:
1. a number of generic events are necessary for the full transition from normal tissue to carcinoma tissue;
2. critical genes, referred to as “gatekeepers”, exist and their mutation/loss represents a point-of-no-return, from which a progression to carcinoma results. An example of a gatekeeper gene is the gene for adenomatous polyposis coli (APC), the mutation/loss of which leads to the development of polypous colon carcinoma;
3. the gene mutations must occur in a specific sequence to lead to a tumor. For example, isolated k-ras mutations, i.e. without a preceding “gatekeeper defect”, result in the apoptosis (programmed cell death) of the affected cells. Conversely, k-ras mutations that follow a gatekeeper defect are permissive and contribute constantly to be phenotype of the tumor. The point in time and persistence of an alteration are therefore also of importance of an alteration in the development of a tumor.

The terminology for the different tumor stages and qualities within the framework of carcinogenesis are important for the assessment of the present invention. A fundamental distinction is drawn between hyperplasia and dysplasia. Hyperplasia is characterized by a pure replication of cells of normal morphological differentiation. In accordance with the prior art, the development of hyperplastic polyps does not result in carcinogenesis. Dysplasia is characterized by the appearance of cells that are qualitatively altered, i.e. are dedifferentiated in different ways. Dysplasia is based on unregulated new development of cells (neoplasia) not consistent with the organ, from which an adenomatous polyp, either a facultative or obligate precancerosis, according to the degree of differentiation, develops. In contrast to hyperplasia, neoplasia is accompanied by a defect in a gatekeeper, as described above. It has not yet been established whether a systematic pathobiochemical link exists between hyperplastic and dysplastic (adenomatous) polyps, as, to date, no change was known that could be demonstrated to the same degree in all forms of morphologically-altered tissue.

The Gatekeeper APC

The initiation of carcinogenesis of most colorectal carcinomas requires the functional loss of the multifunctional APC protein. An important function is the regulation of the β-catenin concentration in the cell. β-catenin is associated with membrane-bound adhesion molecules and migrates between the cell membrane and cell nucleus, where it is responsible for the activity of various genes, via the activation of transcription factors. APC controls the degradation of β-catenin. Defective APC proteins lead to an elevated intracellular β-catenin concentration, in both the cytoplasm and the cell nucleus. This can be demonstrated with standard immunological and molecular biological methods. β-catenin is therefore a surrogate marker for the APC function (Fearnhead et al., Hum. Mol. Gen. 2001, Vol. 10, No.7, pages 721ff).

Well over 700 APC mutations have been described APC mutations have been observed in dysplasias, but not in hyperplastic alterations (cell replication alone without qualitative changes). This is one reason why hyperplastic polyps or hyperplastic “aberrant crypt foci” (ACF, microgrowths) are regarded as harmless.

The CEACAM Family

The CEACAM family comprises 29 genes on chromosome 19q. The glycoproteins are expressed in a large number of tissues. CEACAM-1, CEA (CEACAM-5), CEACAM-6 and CEACAM-7 are mainly found in the large intestine, they are located on the cell surface that faces the lumen of the intestine (apical-luminal localization) and constitute a large part of the glycocalyx there. CEACAM molecules are adhesion molecules that bind to one another and form a highly-organized network in the glycocalyx. Within them, CEACAM-1 maintains, via a cytoplasmatic domain, contact to the internal side of the cell membrane and participates in signal transduction phenomena. Its physiological importance in the colon has not yet been demonstrated.

The expression of the CEACAM is substantially changed in colorectal carcinomas, i.e., it is dysregulated or altered CEACAM-1 and CEACAM-7 expression is considerably lowered or entirely lost, whereas CEA and CEACAM-6 are in part considerably overexpressed (a dysregulation of CEACAM expression is also observed in carcinomas of the esophagus, stomach, mammary glands and lungs). The changes in expression are not restricted to carcinomas. A loss of CEACAM-1 is seen equally frequently (>90% of cases) in colorectal adenomas and, thus, in early forms of dysplasia/neoplasia (Nollau P. et al., (1997), Cancer Res. 57, 2354-57).

The cause of the changes in CEACAM expression is unknown. No mutations in the coding or non-coding regions of the DNA sequence have been reported to date, Specific transcription factors that regulate the tissue-specific expression of CEACAM-1 are not known to date. Similarly, no epigenetic phenomena (methylation of regulatory sequences) have been found.

The physiological importance of the CEACAM molecules has not been unequivocally established. The large number of hypotheses presented in the literature are considered below, taking CEACAM-1 as an example. These have been confirmed through in vitro experiments or derived from clinical observations:

CEACAM-1 has an adhesion function. Cells that have been transfected with the CEACAM-1 gene exhibit a greater adherence to one another than non-transfected control cells (Teixeira, A. M., et al. (1994), Blood 84, 21, 1-219.) The adherence is temperature-dependent and calcium-dependent in a manner described for cadherins (Rojas et al., 1990, Cell Growth Differ. 1, 527-533).

CEACAM-1 further has a receptor function. The murine homologue bgp serves as a receptor for the mouse hepatitis virus (MHV) (Blau et al., 2001; J. Viral, 75, 8173-8186). Human CEACAM-1 expressed on human granulocytes serves as a receptor for the OPA proteins of microbial pathogens such as Salmonella (Chen et al., 1997, J. Exp. Med. 185, 1557-1564).

In tissues that regularly express CEACAM-1 there is a loss in expression at the mRNA and protein levels in the corresponding carcinomas, for example, in colorectal carcinoma (Neumaier et al., 1993, Proc. Natl. Acad. Sci. 90, 10744-10748), An exception is gastric carcinoma, where a tumor regularly has an elevated level of expression compared to normal tissue (Kinugasa et al., 1998, Cancer 76, 148-153).

A comparative gene expression analysis (SAGE) for tumor cell lines shows that CEACAM-1 belongs to those genes whose expression is most frequently and most clearly lost (Zhang et al., 1997, Science 276, 1268-1272). The molecular reasons for the loss of CEACAM-1 have not been established to date.

Adenomas, which may be regarded as precursors of colorectal carcinomas, have, in terms of degree and frequency, a loss of CEACAM-1 expression that is identical to malignant tumors (Nollau et al., 1997, Cancer Res. 57,2354-2357). This shows that a loss of CEACAM-1 expression is an early event in carcinogenesis. Animal models show that tumorigenicity in the tumor transplantation model decreases when the cells have been previously transfected with CEACAM-1 (Fournes et al., 2001; Oncogene 20, 219-230).

Expression of CEACAM-1 within the colon crypts starts in the lower half of the crypts and increases continuously in the direction of the lumen (Frangsmyr et al., 1995, Cancer Res. 55, 2963-2967). No CEACAM-1 production can be demonstrated in the region of the proliferating crypt cells. Furthermore, CEACAM-1 is involved in the morphogenesis of epithelia.

The cytoplasmatic domain of CEACAM-1 is involved in signal transmission (Afar et al., 1992, Biochem. Biophys. Acta 1134, 46-52.). Tyrosine residues and serin and threonin have been identified as phosphorylation substrates. The motifs of the domains that control phosphorylation have been identified as activating and inactivating sequence motifs for kinases and phosphorylases (ITAM motifs/ITIM motifs) (Beauchemin et al., 1997, Oncogene 14, 783-790).

All of the investigations of CEACAM-1 functions to date have used established tumor cell lines or transfectomas as model systems. According to the prior art, nothing is known about the expression behavior of CEACAM-1 and the functional role of CEACAM-1 in tumor stages before gatekeeper mutations, or in hyperplastic lesions (hyperplastic ACF and polyps). Furthermore, no link has been described between hyperplastic lesions and dysplasia. No changes of genes or gene products or changes in the expression patterns of these genes and gene products in hyperplasia are known that are also continued in dysplastic tissue.

The inventor has now succeeded in showing that the expression of CEACAM-1, as a member of the CEACAM family, in hyperplastic lesions is downregulated/lost in the frequency known from dysplasia (adenoma and carcinoma). This is the first demonstration of a direct pathobiochemical connection between hyperplasia and dysplasia. This permits the conclusion that both are individual stages of the same tissue development. In addition, the invention is based on the observation that a loss of apoptosis of the affected tissue is associated with the loss of CEACAM-1 expression in hyperplasia. The causality between CEACAM-1 loss and loss of apoptosis is shown herein, by virtue of the fact that the apoptosis ability is dependent on the degree of expression, and possibly the degree of cross-linkage of CEACAM-1 bound to the cell surfaces.

The object of the present invention is to provide a method for the identification of therapeutically-useful compounds that regulate the expression of CEACAM molecules. These compounds can be used for the prevention of tumors and in particular these compounds can prevent tumors of the gastrointestinal region or induce regression of existing hyperplasia by increasing apoptosis.

A further object of the present invention is to provide a test system for screening of the compounds.

It is furthermore an object of the present invention to use these identified compounds in pharmaceutical compositions for prevention of tumorigenesis in individuals, especially those with a predisposition for tumorigenesis, for example individuals who have a gatekeeper defect on a chromosome.

### SUMMARY OF THE INVENTION

The present invention relates to a method for the identification of compound(s), comprising the steps:
incubation of a sample, that can express one or more genes, or one or more gene products, of the CEACAM family with one or more compounds;
incubation of a second sample, that can express one or more genes, or one or more gene products, of the CEACAM family in the absence of the compound(s):
Comparison of the expression of the gene(s)/gene product(s) of the CEACAM family in the samples.

If necessary, the identification of the compound(s) can also be achieved through a determination of the rate of apoptosis in the samples.

Compounds that can directly or indirectly regulate an alteration of the expression of one or more genes, or one or more gene products, or which contribute to an increase in the rate of apoptosis of the cells, can be used in a regimen that prevents the development of early precursor lesions that can lead to carcinomas, or treats such precursor lesions.

According to the invention, the expression of members of the CEACAM family is used as a parameter to identify substances that influence the expression of antigens. The determination of the rate of apoptosis ran be used in a functional sense to identify compounds that can be used for treatment of the precursor lesions.

Moreover, the present invention provides a rest system and kit that enable compounds to be investigated for their capacity to regulate the dysregulation of the expression of members of the CEACAM family, for example to restore expression to the level of an unaltered cell. Furthermore, the test system allows the investigation of substances that influence the signal cascade via a member of the CEACAM family so that the rate of apoptosis is increased, That is to say, compounds can also be identified that, for example, through cross-linking of CEACAM molecules, contribute to a signal transmission that results in an increase in the rate of apoptosis.

These substances can be used in pharmaceutical compositions that are used in the prophylactic treatment of tumors in the beginning of carcinogenesis.

In addition, the present invention relates to a diagnostic method, comprising the determination of CEACAM expression in test samples. This method permits the identification of precursor lesions at an early stage of tumorgenesis.

The invention is based on the new observations that certain molecules in the CEACAM family are important, in a manner not known to date, for the regulation of apoptosis (programmed cell death), but these molecules are unable to perform this regulation as a result of a disruption of their expression in the earliest tissue changes.

It was surprisingly found that there is a direct relationship between the expression of members of the CEACAM family, such as CEACAM-1 and CEACAM-7, and the sensitivity of colon cells to apoptosis.

### FIGURES

FIG. 1: shows the development of hyperplastic and dysplastic (neoplastic) colorectal tumors in accordance with the concepts of the prior art (after Kinzler K. W. and Vogelstein B. 1996, Cell 87, 159-170; Jen J. et al. 1994. Cancer Res., 54. 5523-5526). Whereas hyperplastic lesions do not lead to a tumor progression because of the absence of gatekeeper mutations, the presence of gatekeeper mutations leads to this dysplastic adenoma-carcinoma sequence.

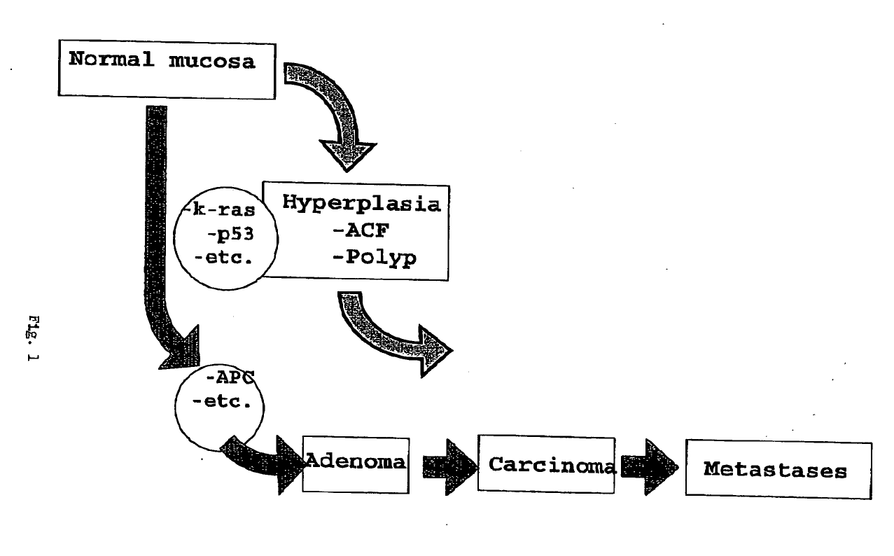

FIG. 2: shows the development of hyperplastic and dysplastic (neoplastic) colorectal tumors in accordance with the findings that form the basis for the invention: A reduction/loss of CEACAM-1 expression occurs in the colon crypt and results in a reduced apoptosis. This leads to hyperplasia of the affected crypt(s) in the form of a so-called aberrant crypt focus (ACF) or hyperplastic polyps (HP). Mutations within ACF or HP can affect different genes. If the mutation affects non-permissive genes (e.g., k-ras) then the hyperplastic tissue will die. In the event of gene mutations that are critical for tumor progression (e.g., gatekeeper or APC), this will lead to a dysplastic change in the sense of new development, representing precancerosis.

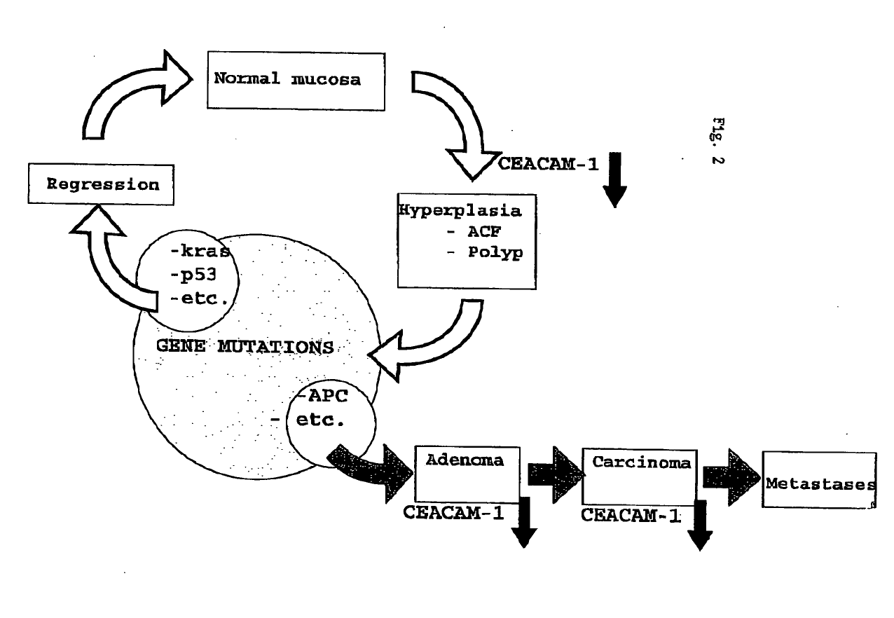

FIG. 3: shows the expression of CEACAM-1 and the accumulation of β-catenin (as the surrogate marker of the APC defect) in lesions that are hyperplastic (ACF, HP) or dysplastic (adenoma=AD, carcinoma=CA). The loss of CEACAM-1 expression is demonstrable at a high and consistent frequency in all tissue changes, whereas APC defects are only seen from the stage of dysplasia (neoplasia) onwards. The result of the common CEACAM 1 alteration suggests a pathobiochemical relationship between dysplasia and hyperplasia.

FIG. 4: shows that the ribozyme-controlled overexpression of CEA leads to apoptosis resistance. Cells that exhibit greater CEA expression react in a less sensitive manner to apoptosis stimuli than interferon-γ. The action of CEA on programmed cell death is thus opposite that of CEACAM1.

FIG. 5: shows the importance of the degree of CEACAM-1 expression and the cross-linking of CEACAM-1 to the cell surface for the triggering of apoptosis in the colon cell line HT29. Addition of gamma-interferon (γIFN) over a short period stimulates HT29 cells to produce CEACAM-1 to various degrees. The cross-linking of CEACAM-1 molecules on the cell surface of activated cells enables specific triggering of apoptosis. Irrelevant antibodies have no effect.

FIG. 6: shows the stimulation of CEACAM-1 expression in HT29 cells after treatment with tumor necrosis factor alpha (TNF-) in a Western Blot.

HT29 colon carcinoma cells were incubated with TNE-(10 ng/ml) for 6 hours. The cells were examined microscopically for vitality, lysed, the whole protein isolated and separated electrophoretically. Lane 1 shows the treated cells, lane 2 the untreated control. 8 μg protein was separated per lane. Detection was performed using a CEA antibody and a peroxidase-labeled anti-mouse secondary antibody in accordance with a standard protocol. The blot was additionally stained for −actin for control of protein charge.

### DETAILED DESCRIPTION OF THE INVENTION

The expressions “CEACAM molecules” and “CEACAM family”, as used herein, comprise all molecules that are regarded as members of the CEACAM family, such as CEACAM-1, CEACAM-6, CEACAM-7 and CEA.

The expression “identification” here means that a compound can be named that has the described characteristics. This comprises the screening of compounds and the subsequent evaluation of the screening results to unequivocally name the compound.

The expression “screening” herein means that a plurality of compounds are investigated with a method. This investigation provides information on specific characteristics of the compounds.

The expression “alteration” or “dysregulation” herein means that the level of the molecule within a cell or on its surface is altered in comparison to the normal situation. For CEACAM-1 this means, for example, that the expression of CEACAM-1 is lowered in the cells in comparison to cells in the normal mucosa. This alteration or dysregulation can be present both at the gene level and at the gene product level.

The method according to the invention is a method for the identification of compounds, comprising the steps:
Incubation of a sample, that can express one or more genes, or one or more gene products, with one or more compounds;
Incubation of a second sample, that can express one or more genes, or one or more gene products, in the absence of the compound(s);
Comparison of the expression of the gene(s) or gene product(s) of the CEACAM family in the samples.

In addition, the invention relates to a method for the identification of compounds, comprising the steps:
Incubation of a sample, that can express one or more genes, or one or more gene products, of the CAECAM family, with one or more compounds;
Incubation of a second sample, that can express one or more genes, or one or more gene products, of the CAECAM family, in the absence of the compound(s);
Comparison of the rate of apoptosis of the cells in the samples.

In particular, the expression of the members CEACAM-1, CEA, CEACAM-6 and CEACAM-7 is determined using the method according to the invention. The degree of expression is related to the increase in the rate of apoptosis of the cells. Furthermore, the method according to the invention allows the detection of compounds that exhibit an ability to influence the signal cascade via CEACAM-1, CEA, CEACAM-6 and CEACAM-7 and thus increase the rate of apoptosis of the cells. That is to say that the method according to the invention also enables compounds to be identified that, upon expression of a member of the CEACAM family, lead to an increase in the rate of apoptosis through cross-linking of said member.

The detection of the test compounds as compounds for the regulation of the alteration of the expression of members of the CEACAM family can be carried out, as for the compounds that bring about a cross-linking of the members, indirectly through determination of the susceptibility to apoptosis, i.e., the rate of apoptosis.

The determination of the rate of apoptosis thus provides information on the efficacy of the compound in influencing the expression of members of the CEACAM family, as well as on the efficacy of the compounds in influencing the signal transduction via the cross-linking of a member of the CEACAM family.

The determination of the susceptibility to apoptosis (apoptosis resistance), i.e., the apoptosis rate, may be carried out through generally-known methods, such as staining with Annexin V or propidium iodide. In addition, methods commercially available, that allow measurement of nucleosomes liberated in the course of apoptosis using a sandwich ELISA, are also suitable. A further method immunochemically measures the caspase-induced proteolysis of cytokeratins and the resultant neoepitope of this filament protein (mAb M30, Roche Molecular Systems).

A beneficial usage of the identified compounds results, in their presence, to an increased readiness to undergo apoptosis compared to the sample incubated in the absence of the compound (the resistance to apoptosis is lowered).

The following methods are given, by way of example, for demonstrating the expression of one or more genes or one or more gene products. These methods are generally established and known to the skilled person.

CEACAM expression can be demonstrated immunohistochemically in situ on tissue sections or in cell cultures through the use of specific antibodies. These may be either monoclonal or polyclonal. Examples of monoclonal antibodies are antibodies to CEA (clone T84.66. C1P83), CEACAM-1 (clone 4D1C2 and clone 29H2), CEACAM-6 (clone Bu33) and CEACAM-7 (clone Bac-2).

The expression can also be demonstrated immunochemically in a quantitative manner in tissue homogenates and cell culture lysates after electrophoretic separation and Western Blot, with the use of the above-mentioned antibodies. Densitometric evaluations of Western Blots can be carried out for this, through which the relative expression of the individual antigens can also be compared to one another.

Finally, the expression can be demonstrated quantitatively by immunochemical methods in tissue homogenates and cell culture lysates. Specific antibodies directed against the corresponding gene products of members of the CAECAM family are used. Suitable methods are standard enzyme immunoassays such as ELISA, RIA, etc.

The expression can also be investigated in situ at the mRNA level. The skilled person can obtain corresponding gene probes from the published sequences of the members of the CEACAM family. The in situ hybridization is by the standard method.

The expression can furthermore be determined semi-quantitatively through analysis of Northern Blots or quantitatively after densitometry. Northern Blots are standard methods and can be performed with corresponding gene probes, obtained from the corresponding sequences of the CEACAM members. The expert can easily obtain and use the applicable gene probes.

The expression can furthermore be determined through amplification methods using CEACAM-specific primer oligonucleotides. The primers can be simply derived from the known sequences and investigated for their specificity. Quantitative methods are available for this and deliver the quantitative results of gene expression (e.g., real-time RT PCR with LightCycler (ROCHF, Molecular Systems) or TaqMan® (Applied Biosystems).

The method for the determination, according to the invention, of the expression of one or more genes, or one or more gene products, is not limited to the above-mentioned. Other methods may also be used that permit a comparison of the expression of members of the CEACAM family in samples in the presence and absence of the compound(s) under investigation.

Array systems that allow evaluation of different factors that influence the CAECAM system are of particular suitability for this. Suitable arrays in principle contain those genes that are involved in the regulation and function of the CEACAM system.

The arrays can be designed for the measurement of mRNA expression (transcription arrays) and the measurement of gene products (protein arrays).

The sample, for which the expression of the gene(s) or gene product(s) in the presence or absence of the test compounds for example, be one of the following:

Cells, for example primary cells or cells altered through genetic engineering. These cells altered through genetic engineering can be cells that can express one or more genes, or one or more gene products, of one or more members of the CEACAM family, by transfecting them with corresponding vectors.

A cell culture line, for example HT29, A818 etc. These cell lines, commercially obtainable from corresponding centers, can be seeded in corresponding quantities, for instance in microtiter plates and are then incubated for a pre-determined time period with and without the test compound.

Furthermore, cell homogenates, or extracts tissues expressing CEACAM-1 can be used, wherein quantitative assays (as given above) can be used for testing of factors that regulate CEACAM-1 expression.

Compounds that can be used in methods according to the invention are not particularly restricted. They may, for example, be:
Peptides, such as oligopeptides or polypeptides, especially also recombinant CEACAM domains.
Proteins, such as cytokines, antibodies and lectins,
Carbohydrates, simple or complex forms
Small molecules, with a molecular weight between 50 and 1000 Da.
Polymers that enable a cross-linking of the individual CEACAM molecules. The material of the polymers is not particularly limited. The main functional characteristics of the polymers can be the repetitive arrangement of the binding domains (e.g., CEACAM domains, binding peptides or cross-linking carbohydrate structures).

These compounds under investigation can be provided in the form of libraries, for instance as used for high throughput investigations. These libraries may also be obtained from commercial sources.

The compounds can be used as such or in the form of their salts.

Compounds that can be identified and selected through the method according to the invention are distinguished by the fact that they can selectively upregulate the expression of CEACAM-1 and/or CEACAM-7, relative to CEACAM-6 and CEA. On the other hand, they can, for instance, downregulate the expression of CEACAM-6 and CEA. The regulation results in increasing the tendency towards apoptosis of the sample (the apoptosis resistance is lowered).

The compounds identified with the method according to the invention can alternatively influence signal transmission through a member of the CEACAM family, for example CEACAM-1, so that the apoptosis rate of the cells is increased.

These compounds according to the invention, identifiable by the method according to the invention, comprise compounds and factors that influence the regulation of the CEACAM molecules. This means that they can be compounds that influence regulating factors, such as transcription factors, and thus lead to an elevated transcription of CEACAM DNA, such as CEACAM-1 and CEACAM-7, or in the case of CEA and CEACAM-6 to a reduced transcription.

It was found in accordance with the invention that:
for example, the expression of CEACAM-1 is lost in most precursors of hyperplastic tumors of the large intestine or is at least greatly reduced. The percentage loss is comparable to that for dysplastic tumor forms known as cancer precursors and seen in carcinomas. It can be concluded from this that the hyperplastic (and intrinsically not dangerous) tumors can develop in a continuous process into dysplastic cancer precursors.
the cross-linking of, for example, CEACAM-1 on the cell surface mediates a specific signal for triggering of apoptosis. This signal is demonstrable in an expression-dependent and cross-linking-dependent manner in the in vitro model in apoptosis-sensitive reporter cells and in human colon cells of line HT29.
in vivo, a CEACAM-1 loss in the investigated hyperplastic tissue lesions correlates with a lowered apoptosis. A lowered apoptosis is regarded as a main cause of the development of hyperplastic colon crypts (Roncucci L. et al. 2000, Cell Prolif. 33, 1-18). The biological function of CEACAM-1 as a means of regulation of the tendency to apoptosis described herein was previously unknown. In so far, the loss described herein by way of example for CEACAM1 is the earliest molecular alteration that is followed by a pathobiochemical alteration in the phenotype of the colon crypts.

According to the invention it has therefore been possible, for the first time, to show a pathophysiologically-relevant link long-sought-after, between hyperplasia (generally regarded as not dangerous) and neoplasia (involved in a progression to carcinomas). The CEACAMs intervene causally in the regulation of apoptosis, and the loss of the signal-issuing CEACAM-1 is the probable causal trigger for the earliest morphologically-recognizable changes in the colon. This is of special importance as CEACAM-1 loss is not a genetically-fixed defect, but rather a functionally-regulative one. Pharmacological intervention strategies that restore CEACAM-1 function are correspondingly ideally suited for a rationally-justified (derived from pathophysiological findings) prevention of hyperplasia.

The results presented above also apply for CEACAM-7. As already described above, the situation is different for CEA and CEACAM-6. With these, the expression of the molecule is upregulated in the altered condition of the cell and the rate of apoptosis thereby lowered. Compounds identified in accordance with the invention are selective for an increase in CEACAM-1 and/or CEACAM-7 and do not lead to an increase in the expression of CEA and/or CEACAM-6. In fact, the compound advantageously leads to a reduction in the expression of CEA and CEACAM-6.

The compounds identified in accordance with the invention can, however, also selectively lead to a lowering of CEA and/or CEACAM-6 and do not lead to a lowering of the expression of CEACAM-1 and/or CEACAM-7. In fact, the compound advantageously leads to an increase in the expression of CEACAM-1 and CEACAM-7.

The compounds identified in accordance with the invention enable the following:
A restoration of, or increase in, CEACAM-1 and/or CEACAM-7 expression, or a reduction in CEA and CEACAM-6 expression, and thus an increase in the ability of the effected crypt cells to undergo apoptosis.
A diminution in the danger of critical mutations through a reduction in hyperplasia.
A usage in the prevention of the tumorgenesis.

Candidate compounds can be tested in vivo in animal models. Test methods in rodents are known for the investigation of carcinogenicity of, for example, substances added to the diet, using an investigation of the resulting ACF frequency in the gastrointestinal tract (Sorensen I. K., 1997, Carcinogenesis 18, 777-781).

Similarly, clinical testing is conceivable in patients who show a predisposition to cancer in the sense of an obligate precancerosis, as is the case with a hereditary defect of an APC allele. The action of compounds that are candidates for prevention can be investigated, e.g., during diagnostic interventions, for example on the basis of the numerical development of colon tumors over time. For this, it is possible to investigate, following biopsy, the gene expression of members of the CEACAM family, in particular CEACAM-1, using the described method.

The test system according to the invention can be an automated test system that carries out the above-mentioned steps for investigation of influencing factors on the CEACAM system. In particular, this may be a test system that is suitable for high throughput analysis.

The kit according to the invention can contain the necessary components for performance of the method according to the invention and includes the sample and all necessary reagents for incubation.

The use of the identified compounds in pharmaceutical compositions is possible in the usual quantities and dosages. The pharmaceutical compositions can be provided in the usual formulations for administration with the selected compound as the effective agent.

Individuals with a predisposition, for whom preventive-therapeutic usage of the identified compounds is particularly advantageous, are those who develop familiar adenomatous polyposis (FAP) or hereditary nonpolyposis colorectal cancer (HNPPC), and risk groups for somatic mutations of gatekeeper genes.

Changes in the expression of CEACAM have also been observed for carcinomas of other tissues, for example the esophagus, stomach, mammary glands and lungs. The compounds identified in accordance with the invention can also be used here.

The diagnostic test method according to the invention can be used for the early diagnosis of precursor lesions. The method comprises the determination of the expression of a member of the CEACAM family, a sample that contains cells of the tissue under investigation and a comparison of expression with that of normal tissue. In particular, this diagnostic method can be a method in which the expression of CEACAM-1 or CEACAM-7 in a sample is determined. The sample is generally a biopsy sample from the test tissue.

Furthermore, a diagnostic kit according to the invention is provided that comprises components for the determination of CEACAM expression in a sample. These components can be primary antibodies directed against a member of the CEACAM family (of either monoclonal or polyclonal origin) that are either directly labeled or not labeled. Furthermore, the kit can contain a secondary antibody directed against the primary antibody and labeled with a marker.

The demonstration can therefore be direct or indirect with known methods, for example immunohistological and immunofluorescent methods. To this end, the antibodies contained in the kit according to the invention to a member of the CEACAM family can be labeled directly with generally-known molecules, including enzymes such as alkaline phosphatase and peroxidase, and fluorescent dyes such as FITC, TRITC, rhodamine, Texas Red, ALEXA® dyes and Cy® dyes. The labeling, however, can also be indirect through the use, of secondary antibodies or antibodies directed against CEACAM that are labeled with molecules such as biotin, digoxigenin or the like and then demonstrated using a secondary reagent.

Furthermore, the kit can contain enzyme substrates for a peroxidase or alkaline phosphatase that permits the enzymatic demonstration of the bound secondary molecule.

### EXAMPLES

### Example 1

### CEACAM-1 Expression in Adenomas of the Colon

Freshly-derived samples of adenomas from patients were investigated for the expression of different CEACAM genes (Nollau P, et al., (1997), Cancer Res. 57, 2354-2357). For this purpose, total RNA was isolated from the samples through a conventional method and analyzed using Northern Blot (Neumaier M. et al., PNAS, (1993), 90 (22), 10744-10748).

The Northern Blots were evaluated using quantitative densitometry and image analysis and the alterations in the expression of CEACAMs compared to the corresponding normal tissue of the same patient (matched-pair analysis). The results for the adenomas are presented in Table 1 below.

TABLE 1

| Sample No. | CEACAM1 expression |
|---|---|
| 8BI | Ø |
| 9BII | ((+)) |
| 13BI | Ø |
| 13BII | ((+)) |
| 14BI | Ø |
| 14BII | ((+)) |
| 15BI | ((+)) |
| 15BII | (+) |
| 18BI | ((+)) |
| 19BII | ((+)) |
| 23BI | Ø |
| 23BII | Ø |
| 24BI | ((+)) |
| 25BI | Ø |
| 25BII | Ø |
| 30B | (+) |
| 31B | (+) |
| 32B | ((+)) |

Ø = Specific CEACAM1 RNA no longer detectable
(+) = 60% loss in expression
((+)) = >80% loss in expression

### Example 2

### Immunohistochemical Demonstration of the Change in CEACAM1 Expression in Different Precursor Lesions

Normal colon mucosa tissue was freshly obtained from surgical interventions. The material was fixed in 4% formaldehyde/PBS at 4° C. for 1 to 4 hours using a standard protocol. The samples were stained with 0.2% methylene blue (Sigma, Taufkirchen, Germany) for 3 minutes and investigated for the presence of ACF using a magnifying glass. Samples with more than 7 foci were used for further investigations. Samples of hyperplasic polyps, adenomas and carcinomas were obtained from the Pathology Institute of the University Hospital in Hamburg for comparison purposes.

5 μm sections were derived from the tissue samples embedded in paraffin by a standard technique. The paraffin was removed from the sections on microscope slides and the sections washed with PBS. The sections were then pre-treated in a microwave for 10 minutes at 650 W in 10 mM citrate buffer (pH 6.0) or for 3 minutes at 650 W in 1 mM EDTA buffer (pH 8.0), followed by 7 minutes at 160 W.

The sections were then stained as follows using the following antibodies:
IMMUNOHISTOCHEMICAL DETECTION OF CEACAM-1. Monoclonal CEACAM1-specific antibody clone 29H2 (Novocastra, Newcastle, UK, diluted 1:50) or clone 4D1.C2 (Prof. C. Wagener, Department of Clinical Chemistry, University Clinic, Hamburg, Germany, 4 μg/ml).
IMMUNOHISTOCHEMICAL DETECTION OF CEA. Monoclonal antibody T84.66 (Prof. J. E. Shively, City of Hope National Cancer Center, Duarte, Calif., USA) against CEA with high specificity; in particular no cross-reaction with CEACAM-1 or CEACAM-6 (5 μg/ml).
IMMUNOHISTOCHEMICAL DETECTION OF APC FUNCTION. Monoclonal antibody 7D11 (Alexis, Grunberg, Germany) against human β-catenin (1 μg/ml).
IMMUNOHISTOCHEMICAL DETECTION OF APOPTOSIS. Monoclonal antibody M30 (ROCHE, Molecular Systems, Mannheim, Germany) against apoptosis-induced caspase-caused neoepitope of human cytokeratin-18.
IMMUNOHISTOCHEMICAL DETECTION OF APOPTOSIS. Monoclonal antibody CH11 (Coulter Immunotech, Heidelberg, Germany) against human CD95 (1 μg/ml).

The sections were incubated with the primary antibodies overnight at 4° C. in moist chambers. Specific peroxidase-based staining was carried out using the VECTOR Elite KIT (Vector Laboratories, Burlington, Calif., USA) using diaminobenzidine in accordance with the instructions of the manufacturer. All washing steps were carried out with PBS (pH 7.4) under strict observance of the instructions of the manufacturer. The immunohistochemical results were evaluated visually semi-quantitatively.

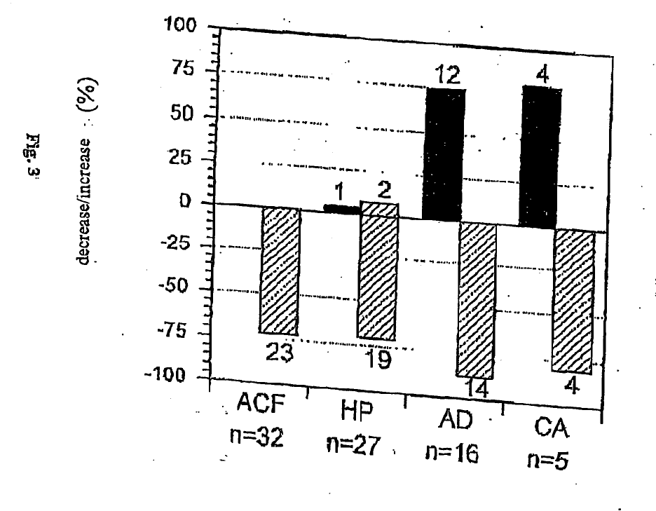

The expression of CEACAM1 and β-catenin (as a measure of the β-catenin accumulation caused by the APC defect) for the tissues investigated is shown in FIG. 3.

The data presented clearly show that a loss of CEACAM-1 expression is already found in the ACF and HP, whereas the surrogate marker β-catenin used for APC does not show any alteration, and is only to be found in neoplasic rumors.

This demonstrates that the alteration in expression of CEACAM-1 represents a markedly earlier event in the development of tumors than the defect in the APC gene, generally regarded in the prior art as the first event for colon carcinoma (accumulation of β-catenin expression). In addition, the loss in CEACAM-1 expression is retained over the entire development of multi-step carcinogenesis. The alteration of CEACAM-1 expression therefore takes place at the hyperplasia stage and is similar to that in advanced neoplasias. By contrast, the accumulation of β-catenin as the surrogate marker for alterations to APC is first demonstrable with neoplasias; hyperplastic changes are not recognized.

### Example 3

### Significance of the Expression of CEA (CEACAM-5) for Apoptosis Sensitivity in HT29 Cells

Ribozyme-regulated mRNA expression is used to investigate the influence of CEA protein expression on the susceptibility of the cells to apoptosis. HT29 colon cancer cells (obtained from ATCC, Rockville, USA) were transfected with CEA-targeted hammerhead ribozyme expression vector.

The vector was derived by a method analogous to that of Schulte et al., PNAS 1996, 93, pages 14759ff and Juhl, H., et al, 1997, JBC 272, pages 29482ff.

The following oligonucleotides were used as ribozyme sense and antisense nucleotides.

| | |
|---|---|
| 5′-agcttTGCTCTTCTGATGAGTCCGTTAGGACGAAACTATGGAgggcc-3′ (sense) | (SEQ ID NO. 1) |
| and | |
| 5′-cTCCATAGTTTCGTCCTAACGGACTCATCAGAAGAGCAa-3′ (antisense) | (SEQ ID NO. 2) |

These were hybridized and ligated in the pTET vector, as described by Schulte et al,, PNAS 1996, 93, pages 14759ff. The plasmid obtained, pTFT/Rz2113, contains CEA-specific flanking regions at the 5 ′ end (7 nucleotides long) and at the 3′ end (8 nucleotides long). These embrace the catalytic core sequence of the hammerhead structure and target these on the B3 domain of CEA.

### Transfection of the HT29 Cells

The cells were first transfected with the vector pUHG15-1 (Gossen and Bujard, PNAS, 1992, 89, pages 5547ff) using the LipofectAmine system (Life Technologies) to obtain the cell clone HT29/tTA-5. This clone exhibited the optimum transactivated tetracycline activity.

This clone was then mixed with the above derived plasmid pTET/Rz2113 (10 μg) and co-transfected with 1 μg pZeo (Invitrogen, San Diego, USA). This allowed derivation of zeocin-resistant clones containing the ribozyme. The clones were selected in culture with 0.4 mg/ml zeocin and 1 μg/ml tetracycline and investigated for regulated CEA expression using FACS analysis (FACStar plus, Becton Dickinson). The HT29 cells were otherwise cultured in IMEM Medium (Life Technologies Inc., Gaithersburg, USA), plus 10% heat-inactivated Fetal Calf Serum and glutamine at 37° C. with 5% CO₂.

A clone HT29/Rz4 was obtained for which CEA expression is regulated in a tetracycline-dependent manner. This regulation was approximately 50% here and in general is at least 30%. preferably at least 50%.

### Determination of Apoptosis

1×10⁶ cells were harvested, washed twice with 300 μl cold PBS, pH 7.4 and stained in 100 μl propidium iodide/Annexin V-FITC double-stain solution (TACS™ Annexin V-FITC protocol, Trevigen, Gaithersburg, USA) in accordance with the instructions of the manufacturer. They were incubated in the dark for 15 minutes at room temperature. 400 μl of 1× concentrated binding buffer was then added to the cell suspension and the cells analyzed within 1 hour using the flow cytometer.

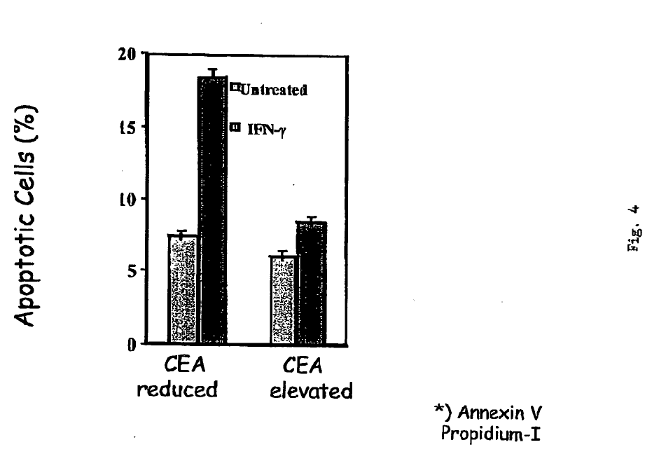

The results of analysis are presented in FIG. 4. It can clearly be seen that the HT29 cells with an increased CEA expression (without addition of tetracycline for ribozyme activation) exhibit a lower rate of apoptosis if the apoptosis is induced through addition of γ-interferon. By contrast, the cells with a lowered CEA expression (following induction of the CEA-specific ribozyme through addition of tetracycline) exhibit a considerably higher sensitivity to apoptosis. This result showed that CEA (CEACAM-5) lowers the capacity to undergo apoptosis in a concentration-dependent and indirect manner. The data show that CEACAM-1 and CEA act antagonistically in HT29 (see below).

### Example 4

### Significance of the Expression of CEACAM-1 for the Apoptosis Sensitivity in Jurkat Cells

The Jurkat cell line is a standard reporter cell line for apoptosis assays. Jurkat cells themselves are CEACAM-negative. To determine the importance of CEACAM-1 for apoptosis, Jurkat cells were transfected with cDNA coding for transmembranous CEACAM-1 by standard methods (BGP-Jurkat). BGP-Jurkat cells express CEACAM-1 on their surface, as can be demonstrated by FACS analyses with CEACAM-1-binding antibodies. Total protein extracts of BGP-Jurkat cells exhibit a regular CEACAM-1 band with a relative molecular weight of approximately 160 kDa in Western Blot.

BGP-Jurkat cells were held under constant cell culture conditions in a logarithmic growth phase and incubated for either 4 or 16 hours with different antibodies. The following were used:
Clone 7D11 (10 μg/ml, against intracellular β-catenin) as negative control,
Clone CH11(100 ng/ml, against human CD95) as positive control,
Clone 4D1.C2 (4μg/ml, specific for human CEACAM-1).

The primary antibodies were added to the medium for 24 hours in each case. After change of the medium, polyclonal goat anti-mouse immunoglobulin (15 μg/ml) was added to the cell culture medium as secondary antibody for cross-linking. No secondary antibody was added to the aliquot with anti-CD95 antibody CH11 (IgM). After a total experiment time of 80-96 hours the cells were stained with trypan blue and propidium iodide using standard protocols and the number of dead/apoptotic cells counted microscopically using blinded evaluation. All test runs were performed in triplicate and the experiments repeated 3 times independently. The results of the individual tests were evaluated statistically using the Wilcoxon signed ranks test.

TABLE 2
Apoptosis sensitivity of CEACAM-1-transfected
and non-transfected Jurkat cells

| | Jurkat | | | | | | |
|---|---|---|---|---|---|---|---|
| | wild-type | | | | | | |
| | | Apoptotic cells | | | CEACAM-1 transfected cells | | |
| | Dead cells (%) | (%) | | | Dead cells (%) | Apoptotic cells (%) | |
| Negative control | 5.11 ± 0.64 | 28.6 | | n.s.^{#} | 5.84 ± 1.39 | 28.2 | |
| Anti-CEACAM-1 | 2.40 ± 0.20 | 30.1 | n.s. | p < 0.05^{#} | 7.86 ± 2.13 | 54.3 | p < 0.001 |
| Anti-CD95 | 88.20 ± 1.30 | 66.4 | p < 0.001 | n.s.^{#} | 89 ± 20.81 | 74.9 | p < 0.001 |

Statistical analysis was performed using the Wilcoxon signed ranks test;
n.s. = not significant;
^{#}Difference in the percentages of dead cells between wild-type Jurkat cells and Jurkat cells transfected with CEACAM-1

Human Jurkat apoptosis reporter cells, previously transfected stably with cDNA coding for transmembranous CEACAM-1, exhibit a markedly-elevated apoptosis if the CEACAM-1 molecules on the surface are specifically bound by the murine monoclonal anti-CEACAM-1 antibody mAb 4D1C2 and cross-linked through an anti-murine secondary antibody. The secondary antibody and an irrelevant monoclonal antibody (mAb 7D11) do not themselves trigger apoptosis. The antibodies have no effect in non-transfected Jurkat cells. As a positive control, the anti CD95 antibody mAb CH11 in the CEACAM-1-expressing and non-expressing cells shows that the Jurkat cells are sensitive to apoptosis. This result shows that CEACAM1 can send an apoptosis signal. Since, aside from CEACAM-1 no other members of the CEACAM family are expressed, the result is attributable specifically to CEACAM-1 and is triggerable through binding and cross-linking by the CEACAM-1 antibody mAb 4D1C2/secondary antibody.

### Example 5

### Significance of the Modulated Expression of CEACAM-1 for the Sensitivity to Apoptosis in HT29 Cells

HT29 cells were treated for 6 hours with 500 U/ml γ-interferon. The procedure was also run without interferon stimulation. After change of medium, the cells were treated with primary antibodies as in Example 4. These were: mAb 4D1C2 (anti-CEACAM-1 at 4 μg/ml) or irrelevant mAb 7D11 (anti-β-catenin at 1 μg/ml). A test run without antibody was used as a second negative control.

After medium change, secondary antibody (anti-mouse at 15 μg/ml) was added. After a total experiment duration of 80-96 hours, the level of apoptosis was determined using standard propidium iodide staining or the apoptosis ELISA of ROCHE Molecule Systems, strictly in accordance with the instructions of the manufacturer. All experiments were performed in triplicate and repeated 3× independently. The Wilcoxon signed ranks test was used to assess the significance of the data.

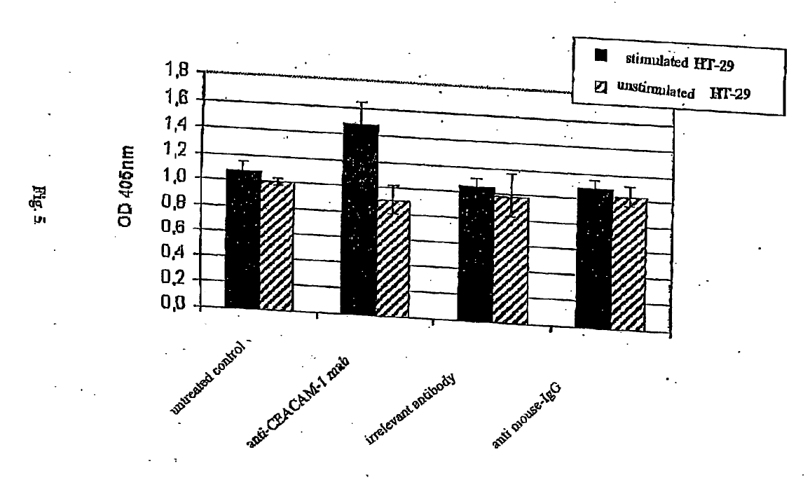

The results (FIG. 5) confirm the importance of CEACAM-1 for apoptosis and the data from the Jurkat reporter cell system.

In addition, the experiment shows that the sensitivity to the triggering of apoptosis is firstly dependent on the degree of CEACAM-1 expression. A pre-requirement, however, is its cross-linking and not just an increase in the number of CEACAM-1 molecules on the cell surface, as the apoptosis in stimulated HT29 (black bar in “untreated control”) shows. Non-specific immunoglobulin-mediated adsorption effects do not lead in stimulated HT29 to a rise in apoptosis (black bars in “irrelevant antibodies” and “anti-mouse IgG”). These rates of apoptosis correspond to those for unstimulated HT29 cells. No increase in apoptosis was observed for the basal low CEACAM-1 expression in HT29, despite cross-linking, whereas in the cells with higher expression an induction of apoptosis was observed.

This experiment shows that, in addition to the degree of CEACAM-1 expression, the cross-linking is of further importance for the apoptosis sensitivity of the HT29 cells. The cross-linking (made possible in vitro through the antibody cross-linking), is ensured in vivo by the presence of the adhesion of molecules of other members of the CEA family in the glycocalyx.

### Example 6

### Specific Upregulation of CEACAM-1 by TNF-α

HT29 colon carcinoma cells were incubated for 6 hours with TNF-α (10 ng/ml). The cells were examined microscopically for vitality, then lysed, their total protein isolated and separated electrophoretically. 8 μg protein was separated per lane. An anti-CEA antibody and an anti-mouse-HRP-coupled secondary antibody (Dianova, Hamburg, Germany) were used for detection. A cross-reacting anti-CEA antibody was chosen to investigate the selectivity of candidate substances for the expression of individual antigens. The selectivity of the substance effects is important since CEACAM-1 and CEACAM-7/CEA and CEACAM-6 can have antagonistic effects on apoptosis.

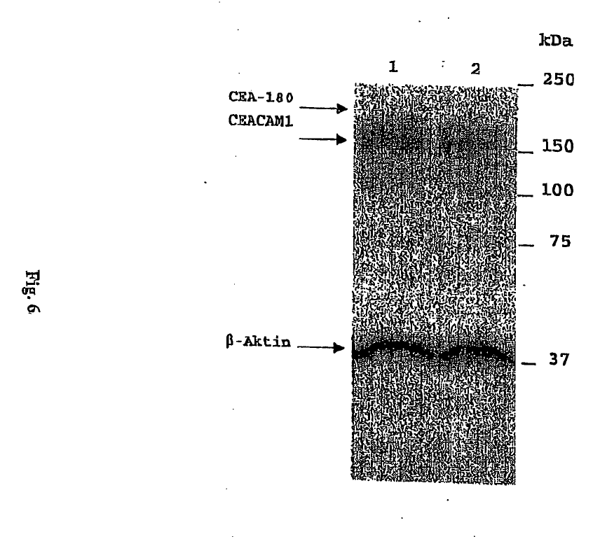

The proportionality of expression of individual CEACAMs can be quantified in Western Blot after densitometric evaluation (e.g., public domain NIH software IMAGE 1.6.1). The antigens are identified in the Western Blot through assignment to relative molar mass. The molecular size of the individual CEACAMs is described in the literature. The Western Blot was normalized with β-actin (Sigma-Aldrich, Taufkirchen, Germany). This blot (FIG. 6) was subsequently evaluated densitometrically using the NIH Image Software 1.6.1.

Treatment with TNF-α resulted in an approximately 2-fold upregulation of CEACAM-1 compared to the untreated sample after 6 hours (lanes 1 and 2). This experiment shows the possibility of specific upregulation of CEACAM-1 expression through exogenously added substances, without influencing other proteins of the CEACAM family.

In contrast to the TNF-α, other substances had no effect on CEACAM-1 expression:

200 nM wortmannin (Calbiochem-Novabiochem, Nottingham, UK) was incubated with 1×10⁶ cells/well (6-well plate) for 3, 8 or 24 hours. After removal of the reagent, the cells were washed with PBS, the total protein fraction isolated and investigated through Western Blot analyses, as described, for changes in CEACAM-1 expression. No changes were found in the expression of members of the CEA family.

5×10⁵ cells were cultured in a T75 flask for 2 days. 3 μg/ml 5-azacytidine (Sigma-Aldrich, Taufkirchen, Germany) were used for the experiment. The medium was changed, with 5-azacytidine (3 μg/ml), on the 3rd day. The medium was changed again on day 4, without 5-azacytidine, with subsequent culture overnight. The medium was changed to one containing 5-azacytidine on day 5, with culture overnight. The medium was changed again on day 6, without 5-azacytidine, with subsequent culture overnight.

Protein was isolated on day 7. The proteins were investigated using Western Blot. An antibody clone mAb C1N3 (Prof. Dr. Kalthoff, Molecular Oncology Department of the Surgical University Clinic, Kiel, Germany) was used at a concentration of 5 μg/ml.

Detection was by means of peroxidase-labeled anti-mouse immunoglobulin using a standard protocol.

The antibody against human β-actin, used for normalization of the signals in the Western Blot, was used at a dilution of 1:10000 (v/v) and the signal subsequently developed according to a standard protocol.

1-33. (canceled)

34. The method for the identification of compounds capable of altering the CEACAM-1 gene or gene product expression, comprising the steps
incubation of a sample, that can express the CEACAM-1 gene or the CEACAM-1 gene product with one of more compounds;
incubation of a second sample, that can express the CEACAM-1 gene or the CEACAM-1 gene product in the absence of the compound(s);
comparison of the expression of the CEACAM-1 gene or gene product in the samples by the measurement of the rate of apoptosis.

35. A method according to claim 34, characterized in that the sample comprises cells or cell lines which are optionally altered by genetic engineering.

36. A method according to claim 34, wherein the compound increases the expression of CEACAM-1 and CEACAM-7 in its presence compared to its absence.

37. A method according to claim 34, wherein the compound increases the expression of CEACAM-1 and CEACAM-7 in its presence compared to its absence and at the same time the compound lowers the expression of CEA and/or CEACAM-6 in its presence compared to its absence.

38. A method according to claim 34, wherein the determination is at the mRNA level.

39. A method according to claim 34, wherein the determination of expression is at the protein level.

40. A method according to claim 34, characterized in that the compounds that cross-link the CEACAM molecules are identified.

41. A method according to claim 40, characterized in that the compounds are antibodies or polymers.

42. A diagnostic method for the recognition of tumor precursor cells, comprising the step of determination of the expression of one member/members of the CEACAM family in a sample.

43. A diagnostic method according to claim 42, wherein the sample is a cell homogenate or a tissue section.

44. A diagnostic method according to claim 42, characterized in that the expression of CEACAM-1, CEACAM-6, CEACAM-7 and/or CEA is determined.

45. A method according to claim 42, characterized in that the lowering of CEACAM-1 and/or CEACAM-7 expression is determined and/or that the increase in CEACAM-6 and/or CEA expression is determined.

46. A diagnostic kit comprising components for the determination of the expression of one member/members of the CEACAM family according to claim 42.

47. Pharmaceutical compositions containing one or more compounds as the effective compound for the prophylatic treatment of tumors.

48. Pharmaceutical compositions according to claim 47, wherein the tumor is a tumor of the gastrointestinal region.

49. Pharmaceutical compositions according to claim 47, wherein the tumor is a colon carcinoma.

50. Method for the prophylatic treatment of tumors comprising the administration of compounds which increases the expression of CEACAM-1 and/or CEACAM-7 in its presence compared to its absence and/or lowers the expression of CEA and/or CEACAM-6 in its presence compared to its absence.

51. The method according to claim 50, wherein the individuals have a predisposition for the development of tumors.

52. Method according to claim 51, wherein the tumor is a tumor of the gastrointestinal region.

53. Method according to claim 51, wherein the tumor is a colon carcinoma. to its absence and/or lowers the expression of CEA


SEQUENCE LISTING (2 sequences)

SEQ ID NO: 1 - DNA, 47 bp, Artificial
  synthetic oligonucleotide
agctttgctcttctgatgagtccgttaggacgaaactatggagggcc

SEQ ID NO: 2 - DNA, 39 bp, Artificial
  synthetic oligonucleotide
ctccatagtttcgtcctaacggactcatcagaagagcaa